(19) United States
(12) Patent Application Publication
(10) Pub. No.: US 2003/0004478 A1
(43) Pub. Date: Jan. 2, 2003

(54) Incontinence-pantieshield for men

(76) Inventor: Lars Mattsson, Hallingsjo (SE)

Correspondence Address: Rolf Fasth, Fasth Law Offices, 189 Curtis Drive, Longview, WA, 98632-5374, US

(21) Appl. No.: 10/182,539

(22) PCT Filed: Nov. 30, 2001
(86) PCT No.: PCT/SE01/02655

(30) Foreign Application Priority Data
Dec. 1, 2000 (SE) 0004454-5

(51) Int. Cl. A61F 5/44; A61F 13/15; A61F 13/20

(52) U.S. Cl. 604/349; 604/385.03; 604/387

(57) ABSTRACT

Incontinence pouch, male pantieshield. Its flat shape enables it to be stored in a pocket from which it can be retrieved and applied with one hand, such that it automatically grips the penis above the glans and is retained in place.

The present invention relates to an optimally embodied, absorbent incontinence pouch for men, which can be applied directly to the penis and is intended to be able to be used as a male pantieshield. This new male pouch is embodied such that as a pantieshield, it can be stored in a pocket and the user can, even with his penis protruding from his flies, retrieve and apply this pouch, which automatically gaps the penis around and above the glans and is retained in place.

The purpose of the present invention is the eliminate the know drawbacks that impair existing incontinence pouches and diapers as regards daily use other than in nursing institutions. The institutional market for incontinence shields for men and women has generally been canvassed in the past twenty years, while a market introduction is the countries of Southern Europe is of recent date. As a consumer product, female incontinence shields have been a success while those for men have had none at all. Sanitary towels as a specific market offer no comparison in this context. However, it is of interest to note that the panty liner market for women underwent a consumer market introduction some twenty years ago and now achieves global sales in the billions. A comparable market for a male pantieshield has not been penetrated for the lack of a functionally usable pouch. A consumer market introduction for this niche can thus only be achieved with an easily applicable male pouch according to the invention and adapted to the consumer market.

In Europe, specially embodied incontinence pouches for men are manufactured by SCA-Hygiene (Tena for men), Coloplas AS (Conveen), Ontex (Euron Microflex Men),Paul Hartmann (Molimed M) et alia. Their common feature is that they possess a pouch-type embodiment, where the penis or the penis and scrotum is inserted into the pouch aperture- A variety of patented detailed embodiments exist. In Europe, a number of these manufacturers have attempted to launch their incontinence pouches as a consumer product with not entirely successful results. In the USA, “guards” are the primary incontinence protection for men and are identical with those for women with “stretch tapes” at the sides. In the USA, too, health care is the dominant market and likewise the products do not achieve any success as consumer products as “this market is based on value and price.”

Other currently known SAP-laminate urine-absorbing pouches are intended for major urine intakes and are optimized for bed-ridden users. They have a pouch shape where the penis is inserted into an aperture in one side of the pouch. U.S. Pat. No. 6,209,142 B1 displays a partial recess located in a folded and laterally sealed pouch side, which recess is disposed by means of an elastic membrane to adapt to and enclose the penis. Patent application EP 0997126 A2 also displays a partial recess with an elastic located section without any contact with the absorbency pad in the longitudinal seal of one of the triangular pouch sides of a Tetra pack between the wide sealed top of the triangular pouch side and the pointed bottom.

U.S. Pat. No. 6,209,142 and EP 0997126 A2 thereby exhibit diametrical invention criteria with regard to the present invention whose sealed top aperture of the pouch halves comprises a flexible resilient device that is seated on or integrated with the absorbency pad, forming during compression an aperture adapted for the insertion of the penis, after which the resilient device reverts and straightens itself and by means of the absorbency pad wholly or partially grips and encloses the penis behind the glans more gently and flexibly. These patent criteria allow an application of the pouch with one hand and are achieved in accordance with the invention in that the elastically resilient device is enclosed in the entire width of the top aperture of the sealed pouch halves, for which reason the projected cross-sectional area around the penis is minimized and even with the penis emerging from the flies, the user can retrieve, apply, and unite pouch and penis, applied corporeally transversally, for which reason non-disturbing longitudinal pouch surfaces affect user mobility. I.e., it is only with a pouch in accordance with the invention that the user obtains optimum application reliability in both body motion and in a sedentary position without the pouch being affected by longitudinal shear or tensile forces.

The invention thus also functions on men with a retracted penis since the flexible resilient device flexibly grips and encloses the penis behind the glans wholly or partly, whereby it holds out the penis and prevents it from assuming a retracted position. The commercially available incontinence pouches on the market today are designed for two distinct user groups: those that solely enclose the penis for men with a normal penis and those for enclosing both the penis and scrotum, for men with a retracted penis. The present invention is the first where a pouch for both user groups is available, also because the pouch is not affected by longitudinal shear or tensile forces.

In relation to U.S. Pat. No. 6,209,142 and above all EP 0997126 A2, the present invention is also less bulky since it is flat and easier for the user to apply since corporeal transversal applications allow a larger (wider) aperture. The present invention should also be easier to manufacture since the aperture recess is thus not transversal in relation to the absorbency pad, i.e., in terms of manufacturing technology all the constituent parts of the invention coincide in one plane instead of 2 or 3, 3 X, Y and Z planes.

A further benefit of the invention is that it can be embodied with the absorbency pad integrating a resilient device so that when it reverts, the penis is compressed such that the urethra is partly closed. In this manner one can achieve a urine flow more adapted to the absorbency pad without any overfilling occurring in acute cases, i.e., a gentler absorbency pad thus implies that one obtains a greater pressure distribution in order to maintain secure retention without fully closing the urethra.

There are currently also penis clamps on the market for which patent applications have been both submitted and not, i.e., some form of device to compress the penis and close the urethra. In general the penis is regulated and its circumference diminished by a Velcro closure, which must be opened in order to urinate.

The present invention also provides for a possibly easily regulated penis clamp closing of the urethra but integrated into an absorbent urine collection pouch. In addition, the resilient device that grips the penis and encloses the urethra during compression generates an adaptable and momentary aperture for regulated urine evacuation.

There are very strong indications in the form of enquiries from well-regarded institutional and consumer-oriented distributors that the latter could successfully introduce on the consumer market this new male incontinence pouch—pantieshields/briefs liner. The market interest is explicitly clear since incontinence problems also affect a large user group who suffer minor spillage before distinct incontinence is present. Our pouch also solves this male problem in accordance with the patent criteria.

The invention will now be described hereinafter in greater detail with reference to the enclosed drawings where

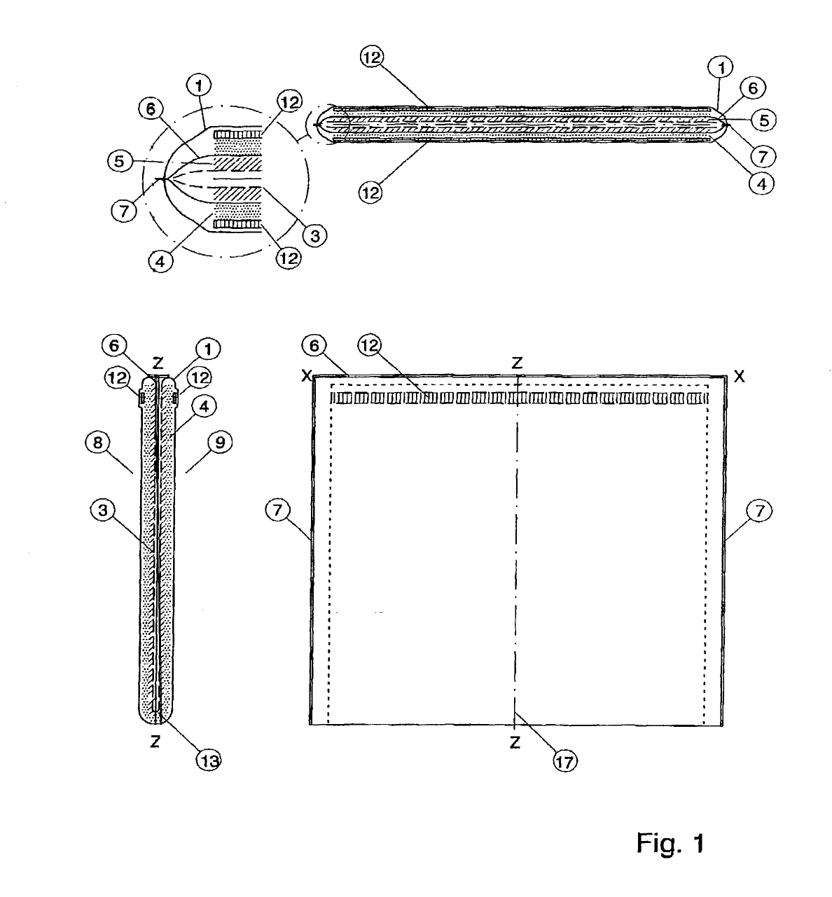

FIG. 1 shows in the American view location a form of the pouch in a flat embodiment.

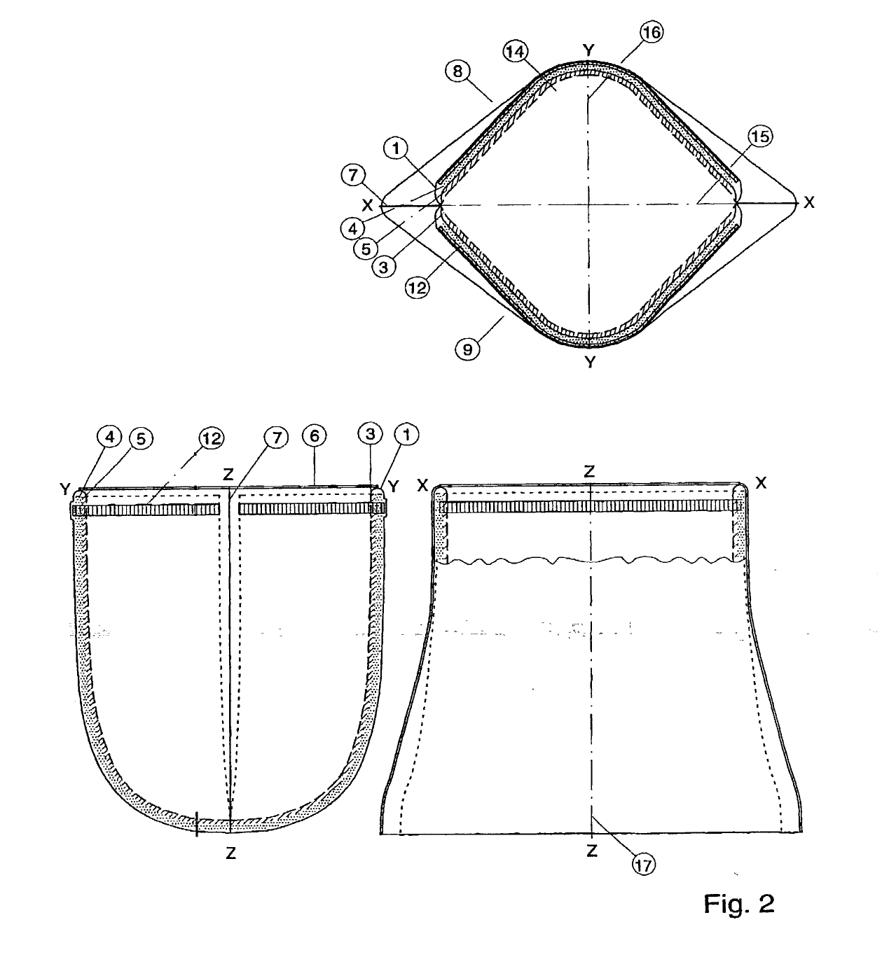

FIG. 2 shows in the American view location the form as shown in. FIG. 1 with a compressed aperture.

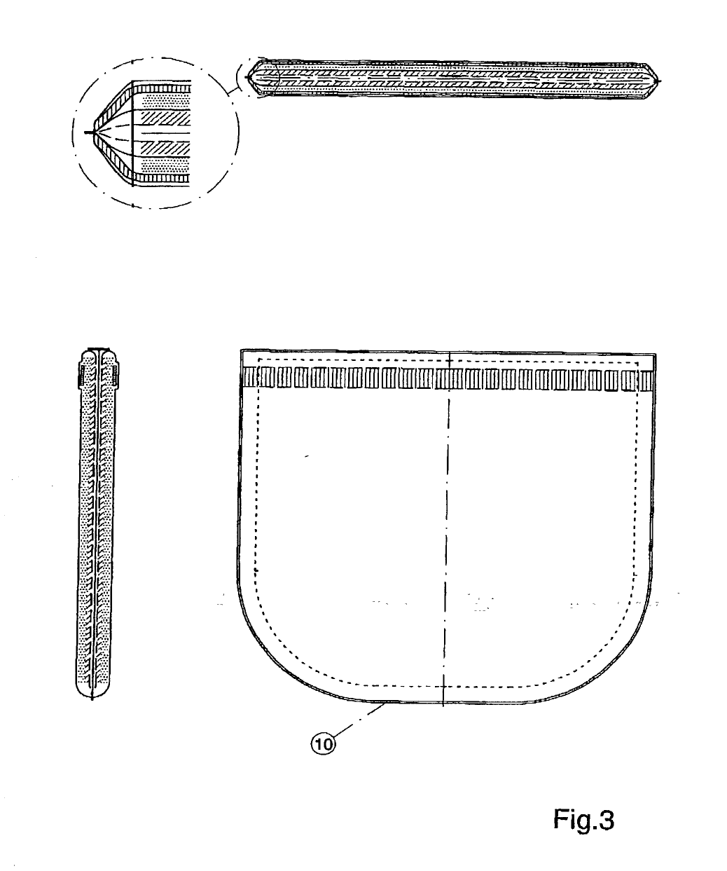

FIG. 3 shows in the American view location a form of the pouch in a flat embodiment.

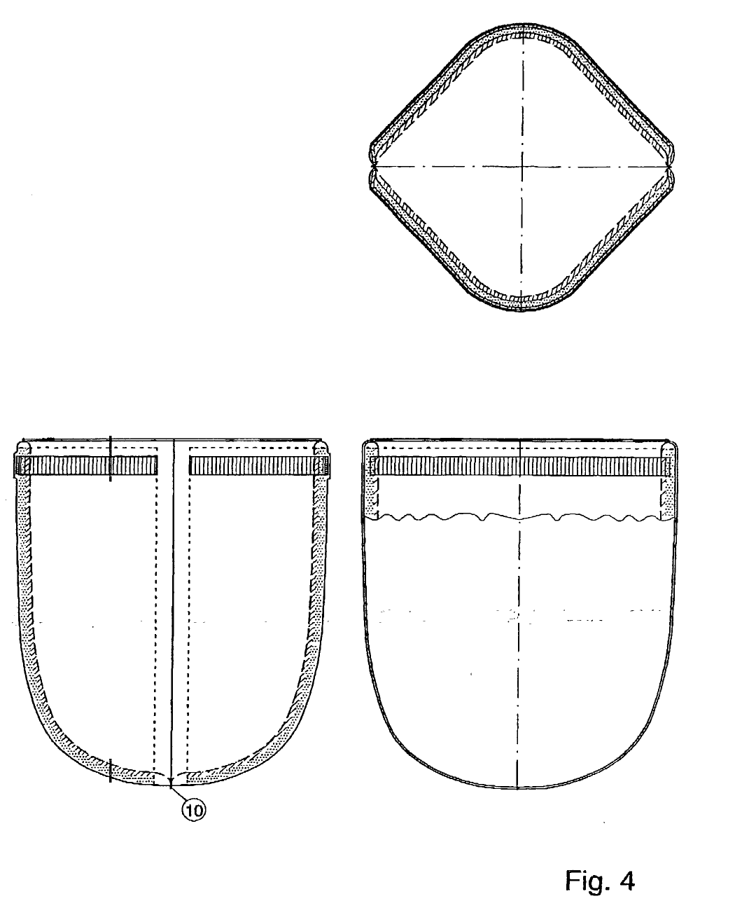

FIG. 4 shows in the American view location the form as shown in. FIG. 3 with a compressed aperture.

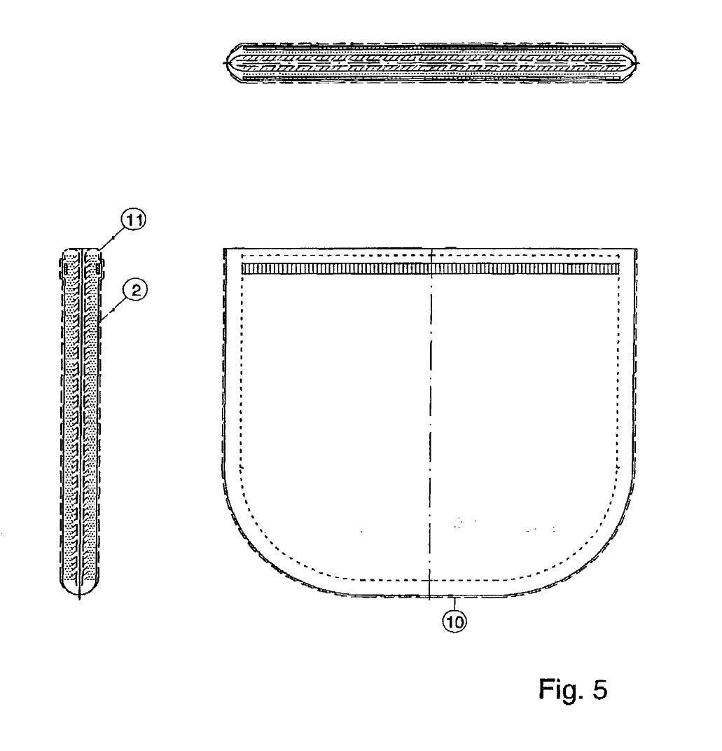

FIG. 5 shows in the American view location a form of the pouch in a flat embodiment.

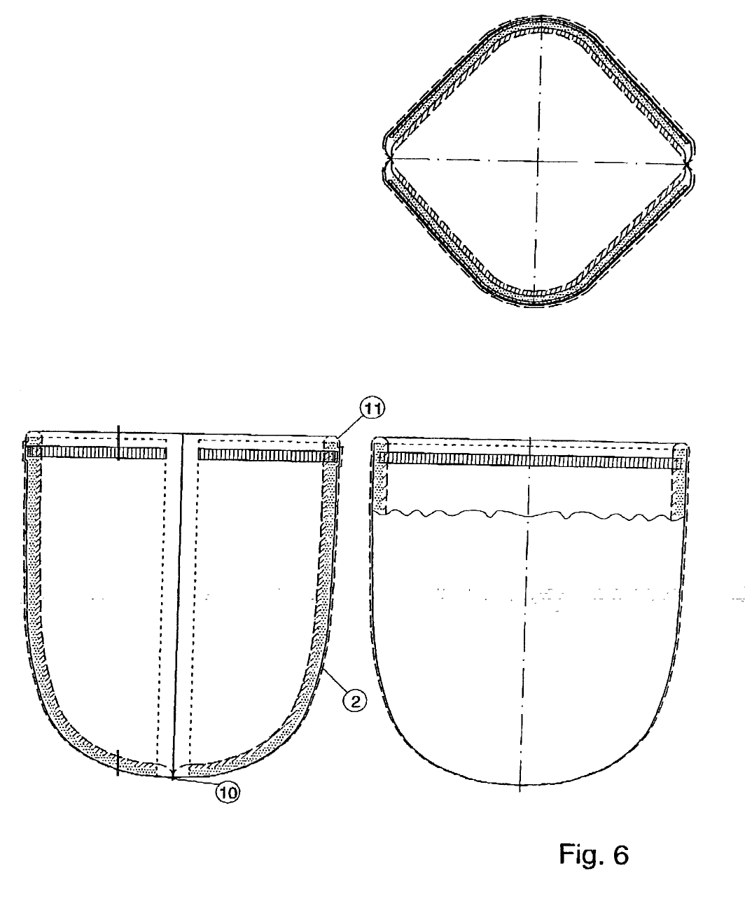

FIG. 6 shows in the American view location the form as shown in. FIG. 5 with a compressed aperture.

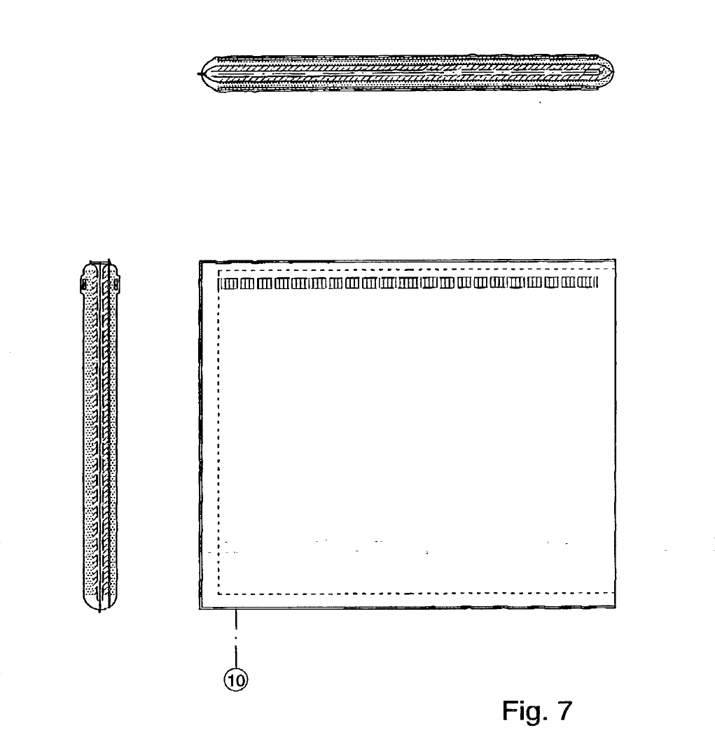

FIG. 7 shows in the American view location a form of the pouch in a flat embodiment.

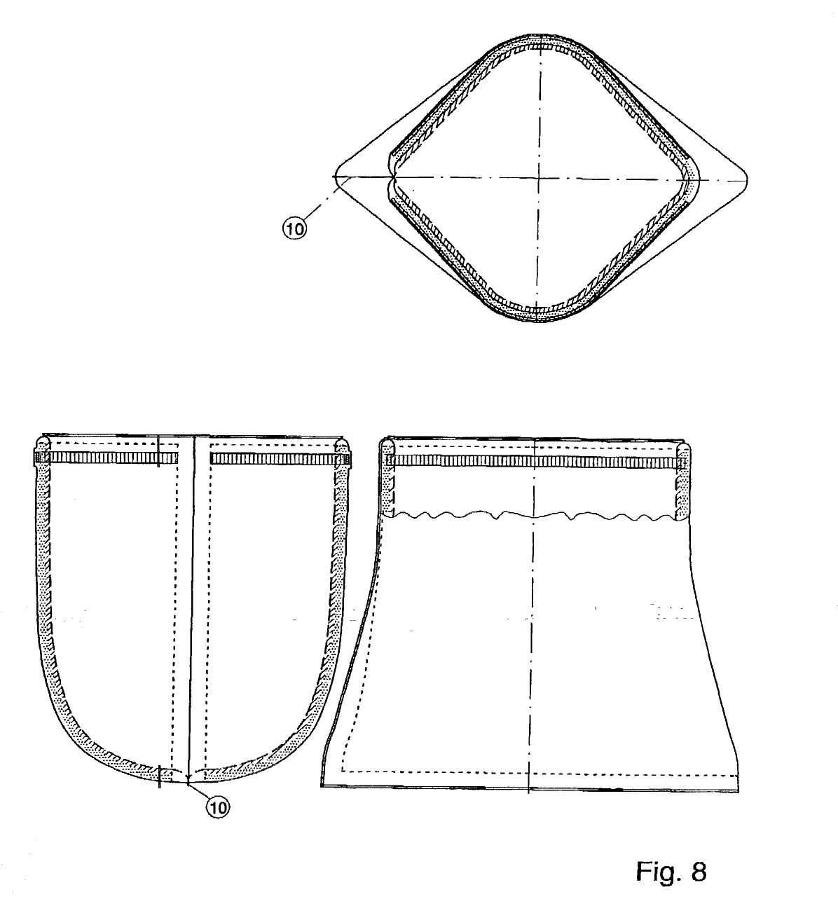

FIG. 8 shows in the American view location the form as shown in. FIG. 7 with a compressed aperture.

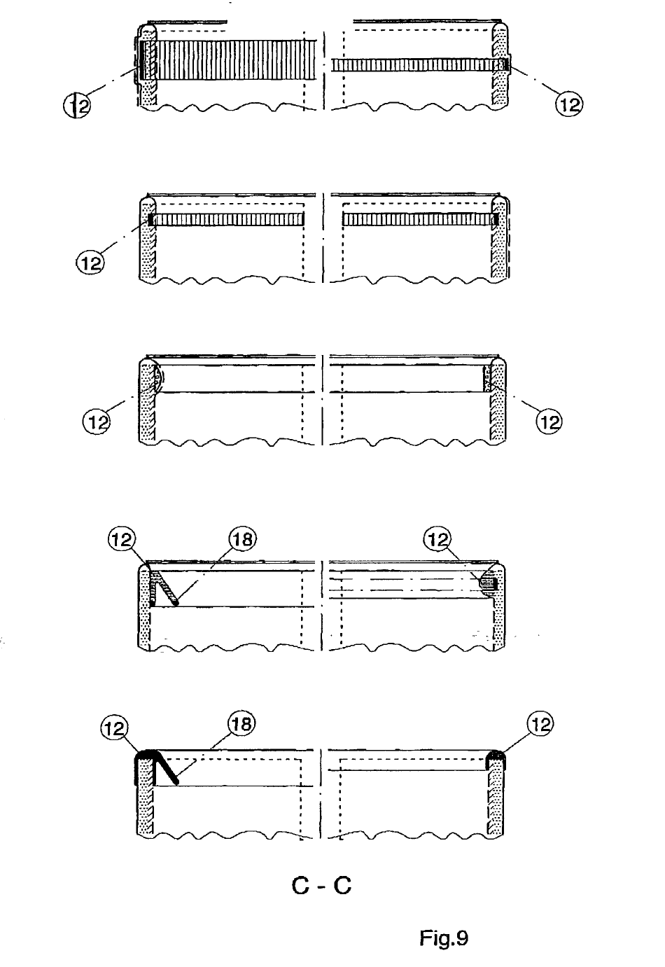

FIG. 9 shows cross-sectional examples of various conceivable resilient device locations

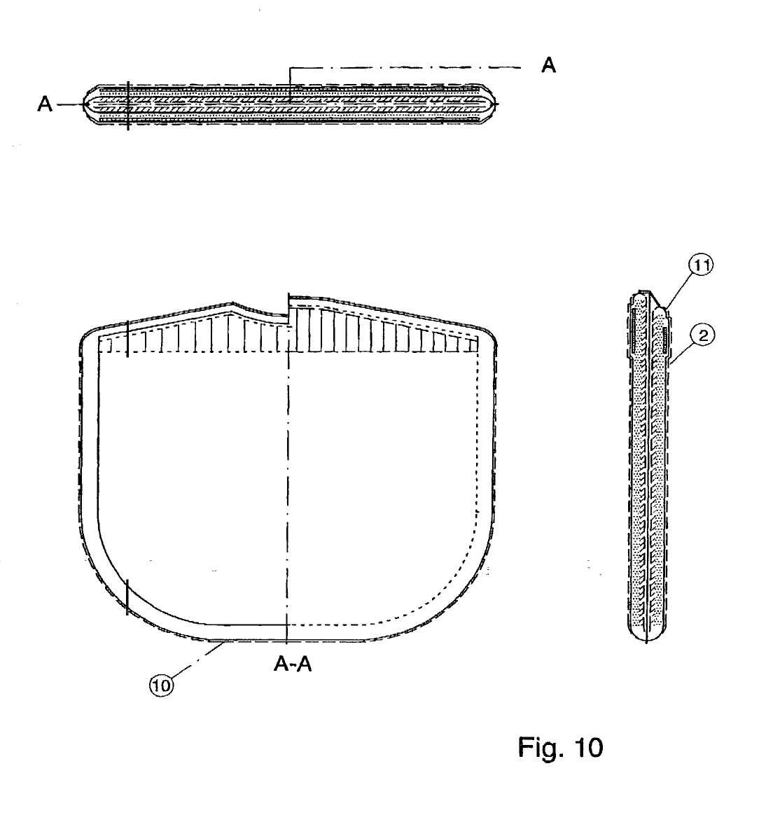

FIG. 10 shows in the American view location a form of the pouch in a flat embodiment.

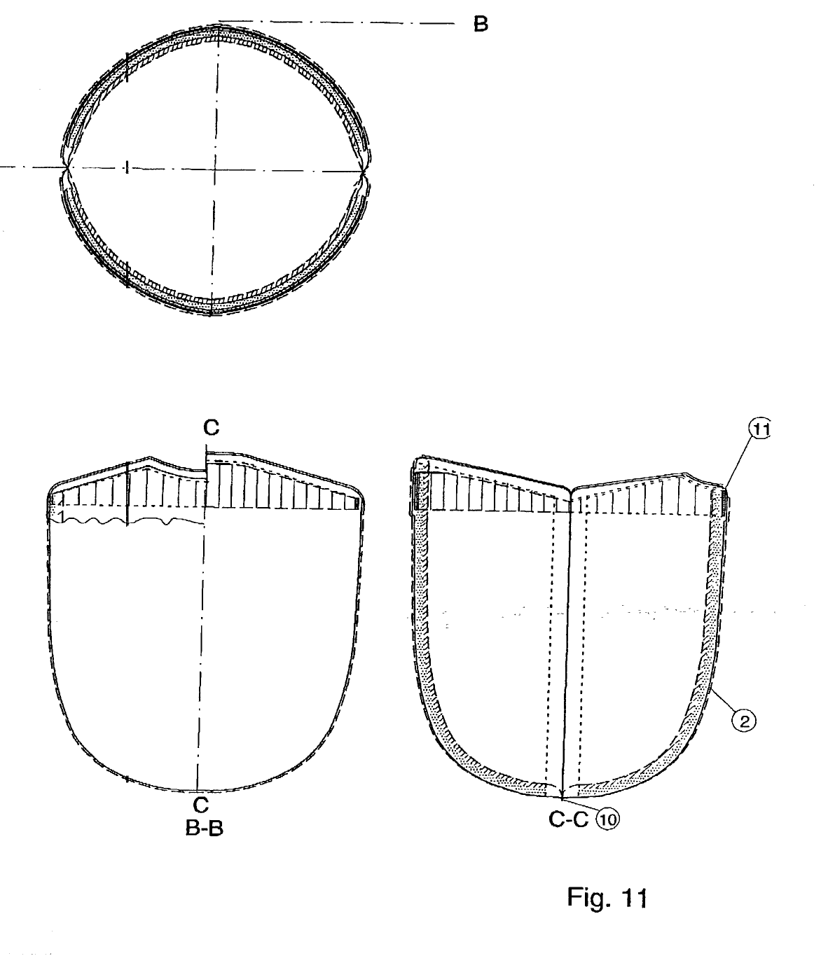

FIG. 11 shows in the American view location the form as shown in. FIG. 10 with a compressed aperture.

The incontinence pouch shown in FIGS. 1-2 comprises a liquid-impervious material, e.g., a PE-film layer (1) that may have been provided on its exterior with a laminated coverstock material preferably a nonwoven (2), an inner liquid-permeable material (3) preferable a thicker nonwoven (3), an absorption layer (4) with or without a diffusion layer (5) and/or a separate diffusion layer (5) applied between the transverse-sealed (6) PE-film layer (1) and the nonwoven (3), which form a pouch shape in that with e.g. a transverse fold (13) and lateral seal (7) of the inner nonwoven (3) to the inner nonwoven (3) with the PE-film layer (1) and the outer nonwoven (2), the pouch halves (8) and (9) are shown sealed as a pouch.

The pouch shown in FIGS. 3-4 presents a pouch shape in that the separately transversally sealed upper pouch half (8) and lower pouch half (9) with a lateral seal (7) and bottom seal (10) of the inner nonwoven (3) to the inner nonwoven (3) with the PE-film layer (1) and the outer non woven (2), the pouch halves (8) and (9) are shown sealed as a U-shaped pouch.

The pouch shown in FIGS. 5-6 presents a pouch shape in that the separately overlap—folded (11) upper pouch half (8) and lower pouch half (9) with a lateral seal (7) and bottom seal (10) of the inner nonwoven (3) to inner nonwoven (3) with the PE-film layer (1) and the outer nonwoven (2), the pouch halves (8) and (9) are shown sealed as a U-shaped pouch.

The pouch shown in FIGS. 7-8 presents a pouch shape in that the transversally sealed (6) upper pouch half (8) and lower pouch half (9) with a lateral seal (7) and bottom seal (10) of the inner nonwoven (3) to the inner nonwoven (3) with the PE-film layer (1) and outer nonwoven (2), which in, e.g., lateral folding show the pouch halves (8) and (9) sealed as a L-shaped pouch.

The pouch shown in FIGS. 10-11 presents a pouch shape in that the separately transversally sealed upper pouch half (8) and lower pouch half (9) with a lateral seal (7) and bottom seal (10) of the inner nonwoven (3) to the inner nonwoven (3) with the PE-film layer (1) and outer nonwoven (2), the pouch halves (8) and (9) are shown sealed as a U-shaped pouch. The pouch shows a good configurative shape with regard to functionality and corporeal effect on the pouch during motion, etc.

The pouches exemplified in FIGS. 1-11 are comprised by a flexibly resilient device (12) exemplified in FIG. 9 and radially applied in the pouch aperture of each pouch half (8) and (9). The resilient device (12) shown can thus be located and applied on, between or in one of the layers (1-5) in the upper pouch half (8) and lower pouch half (9) respectively.

During compression of the ends of the resilient device (12) at the aperture of the incontinence pouch in the pouch-wide X-axis (15) there is formed a circular-shaped aperture (14) in that the Y-axis (16) of the aperture height increases adaptively in order to insert the penis into the Z-axis (17) of the pouch length. When this has been done, the grip is thereby released and the resilient device (12) strives to straighten itself out and the pouch half (8) and (9) will then wholly or partly enclose the penis behind the glans.

The resilient device (12) may comprise, e.g., a spring of steel, bronze, plastic, rubber or other types of known resilient materials. The embodiment of the resilient device (12) can be varied in many ways within the framework of the claims, i.e., other conceivable forms are, e.g., conventional circular springs, plastic or rubber tubes or rods with resilient properties. In addition, other material structures of a softer fibrous nature can be considered since this would then provide in one of the layers an integrated resilient function. Theoretically, the resilient device (12) can also be place don the inside or outside of the pouch halves (8) and (9). If it is placed on the inside it can be comprised, e.g., of a combination of a leaf spring and a softer fibrous material.

In the longitudinal direction of the resilient device (12) the cross-section can be varied and adapted so as to optimize the opening and enclosing characteristics. The resilient device (12) can, e.g., be embodied with some degree of upward bending, whereby compression of the ends of the resilient device (12) at the aperture of the incontinence pouch can more easily open the incontinence pouch aperture in accordance with FIGS. 2,4,6,8. The embodiments of the configuration and hardness characteristic of the resilient device (12) can be freely varied in all dimensions, FIGS. 5 and 6 showing a tapered width configuration outwards that gives a rounder pouch aperture when compressed in the X-axis. The configuration of the resilient device (12) can also in accordance with the example in FIGS. 5 and 6 be embodied such that the resilient device (12) of the lower pouch half by the lower base of the penis is made narrower or configured narrower in the middle. Alternatively, the resilient device (12) of the lower pouch half can be dispensed with altogether or vice versa.

Besides the width configuration, thickness configuration is also conceivable, i.e., the thickness is, e.g., tapered outwards or a combination of width and thickness configuration, relative to the upper and lower sides. In other words, the desired functional characteristics of the resilient device (12) thereby determine its physical embodiment.

A further form of the configuration of the resilient device is shown in FIG. 9, where a lip (18) prevents the penis from being withdrawn from the aperture during motion in that the outer edge of the lip moves radially rearwards and prevents the penis from sliding out.

The aforementioned examples of an incontinence pouch—pantieshield are intended to be applied corporeally transversally. The pouch shapes shown on the drawings can also be varied such that linearity and contour configuration is allowed a free choice of embodiment. Furthermore the location of the position of the resilient device (12) in its depth dimension is determined by such criteria as easy applicability and absorbent capacity. The upper pouch half (8) and lower pouch half (9), too, can have different lengths where there is a requirement for, e.g., overlapping flaps on the upper pouch half (8). The longitudinal sides of the pouch halves can have folded-in or overlapping lateral flaps with or without overlapping flaps etc.

In order to optimize reliable fixation of the pouch, there can of course be applied an “underpants fixation adhesive” with associated release paper or so-called double-sided adhesive tape externally on and towards the underpants and the PE-film layer (1) or coverstock material (2).

The invention is naturally not limited to the forms shown or exemplified on the drawings and these can be varied in many ways within the framework of the claims.

1. An incontinence pouch for men comprising a liquid-impervious material, e.g., a PE-film layer (1) that may have been provided on its exterior with a laminated coverstock material preferably a nonwoven (2), an inner liquid-pervious material (3) preferable a thicker nonwoven (3), an absorption layer (4) with or without a diffusion layer (5) and/or a separate diffusion layer (5) applied between the PE-film layer (1) and the nonwoven (3), which form a pouch shape in that in scaling the inner nonwoven (3) to inner nonwoven (3) with the PE-film layer (1) and outer nonwoven (2) the upper pouch half (8) and the lower pouch half (9) display an aperture characterized in that in the aperture formed in each of the flat pouch halves (8) and (9) facing each other there is applied a resilent device (12) which during compression of the outer ends of the resilent device (12) at the aperture of the pouch width, forms a circular pouch aperture (14) which is applied corporeally on the width and where the penis is inserted into the longitudinal direction of the pouch, whereby the grip is released on the outer ends of the resilent device (12) of each pouch half and the pouch halves (8) and (9) will, when the resilient device (12) returns, strighten themselves and thereby wholly or partly enclose the penis, whereby the incontinence pouch shield is retained in place and the clamping center of the resilient device conincides with the upper and lower side of the penis at its base.

2. Incontinence pouch for men as claimed in claim 1 characterized in that spring device (12) is applied parallel to the aperture plane between any or some of the PE-film layers (1) of the pouch halves (8) and (9) PE-film layer (1) with or without a fixed or separate coverstock (2) and inner liquid-pervious material (3) but placed on the outside of the enclosed absorption layer (4) with or without a fixed or separate diffusion layer (5).

3. Incontinence pouch for men as claimed in claim 1 characterized in that spring device (12) is applied parallel to the aperture plane between any or some of the PE-film layers (1) of the pouch halves (8) and (9) PE-film layer (1) with or without a fixed or separate coverstock (2) and inner liquid-pervious material (3) but placed between the enclosed absorption layer (4), with or without a fixed or diffusion layer (5), and the separate diffusion layer (5).

4. Incontinence pouch for men as claimed in claim 1 characterized in that spring device (12) is applied parallel to the aperture plane between any or some of the PE-film layers (1) of the pouch halves (8) and (9) PE-film layer (1) with or without a fixed or separate coverstock (2) and inner liquid-pervious material (3) but placed on the inside of the enclosed absorption layer (4) with or without a fixed diffusion layer (5).

5. Incontinence pouch for men as claimed in claim 1 characterized in that spring device (12) is applied parallel to the aperture plane below any or some of the PE-film layers (1) of the pouch halves (8) and (9) PE-film layer (1) with or without a fixed or separate coverstock (2), inner liquid-pervious material (3) and enclosed absorption layer (4) with or without a fixed or separate diffusion layer (5), internally on the liquid-impervious material (3).

6. Incontinence pouch for men as claimed in claim 1 characterized in that spring device (12) is applied parallel to the aperture plane above any or some of the PE-film layers (1) of the pouch halves (8) and (9) PE-film layer (1) with or without a fixed or separate coverstock (2), inner liquid-pervious material (3) and enclosed absorption layer (4) with or without a fixed or separate diffusion layer (5), externally on the PE-film layer (1) with or without a fixed or separate coverstock (2).

7. Incontinence pouch for men as claimed in claim 1 characterized in that spring device (12) is applied parallel to the aperture plane above any or some of the PE-film layers (1) of the pouch halves (8) and (9) PE-film layer (1) with or without a fixed or separate coverstock (2), inner liquid-pervious material (3) and enclosed absorption layer (4) with or without a fixed or separate diffusion layer (5), externally on the PE-film layer (1) with or without a fixed or coverstock (2) but between an external horizontal separate coverstock layer (2).

8. Incontinence pouch for men as claimed in claim 1 characterized in that spring device (12) is applied enclosing any or some of the aperture edge surfaces of the pouch halves (8) and (9), including the PE-film layer (1) with or without a fixed or separate coverstock (2), inner liquid-pervious material (3) and enclosed absorption layer (4) with or without a fixed or separate diffusion layer (5).

9. Incontinence pouch for men as claimed in claims 3,4,5 and 8 characterized in that the spring device (12) is provided with a lip (18) articulated, angled and directed inwards and downwards towards the bottom (10), in all or part of the pouch width in the X-axis of the spring device (12).
